(19) United States
(12) Patent Application Publication
(10) Pub. No.: US 2016/0302671 A1
(43) Pub. Date: Oct. 20, 2016

(54) Prediction of Health Status from Physiological Data

(71) Applicant: Microsoft Technology Licensing, LLC, Redmond, WA (US)

(72) Inventors: Farah Shariff, Kirkland, WA (US); Zongyi Liu, Issaquah, WA (US); Dake Sun, Redmond, WA (US); Haithem Albadawi, Redmond, WA (US)

(21) Appl. No.: 14/688,934

(22) Filed: Apr. 16, 2015

(51) Int. Cl. A61B 5/0205; A61B 5/01; A61B 5/00; A61B 5/024; A61B 5/08

(52) U.S. Cl. CPC A61B 5/0205; A61B 5/02427; A61B 5/0809; A61B 5/681; A61B 5/0022; A61B 5/7267; A61B 5/7246; A61B 5/01

(57) ABSTRACT

Collection and analysis of physiological reading can predict when a person is likely to develop a fever before that person's body temperature increases. In an implementation a device such as a wearable band collects physiological information from its wearer. The physiological information may include heart rate or respiration rate. The person's physiological information classified by an algorithm derived through machine learning techniques. The algorithm may be trained by using data from other individuals who are both healthy and who are sick and/or trained from past reading of the person's own physiological readings. The algorithm may evaluate a value of the person's physiological information to generate probabilities that the person is healthy or that the person likely to become sick and/or develop a fever in the next few days.

### BACKGROUND

Many people are interested in knowing more about their health. Advances in computing such as processing speeds, miniaturization, and storage capacity have led to new ways for computers to be used in healthcare. Large amounts of health-related data may be stored and analyzed by computers. Computers may be put in wearable devices that can collect information about the health of a wearer. These and other advances may be used to provide greater insight into health and illness.

### SUMMARY

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

Aspects of an individual's health status may be predicted based on certain physiological data. For example, core temperature rises when a person has a fever. The presence of a fever is usually related to stimulation of the body's immune response. Fever is generally detected by measuring core temperature either directly or from an external surface of the body. Other physiological changes in the body may precede an increase in core temperature. For example, many people when they are about to get sick, start feeling malaise (i.e., tired lethargic feeling) before their temperatures rise. Although malaise may be difficult to measure, identification of the appropriate metrics and appropriate analysis of those metrics can provide a tool to predict when a person is likely to develop a fever before core temperature actually rises. With this knowledge people can proactively take actions to maintain health and support their immune systems.

Physiological data that may be indicative of impending fever can be collected from one or more sensors located on a person's body. The sensor(s) may be placed in a wearable device such as a wrist band. The physiological data that is collected by the sensor(s) can include heart rate, respiration rate, and/or other data. Data collected from the sensor(s) may be stored and analyzed to determine the health status (i.e., healthy or sick) of a person.

### DESCRIPTION OF THE DRAWINGS

The Detailed Description is set forth with reference to the accompanying figures. In the figures, the left-most digit(s) of a reference number identifies the figure in which the reference number first appears. The use of the same reference numbers in different figures indicates similar or identical items.

FIG. 1 shows an illustrative architecture for collecting and analyzing physiological data.

FIG. 2 shows an illustrative block diagram of the computing device from FIG. 1.

FIG. 3 shows an illustrative process for classifying the health status of a patient.

FIG. 4 shows an illustrative process for assigning a patient to a group based on a model generated by machine learning.

FIG. 5 shows an illustrative process for assigning a patient to a group based on a model generated by machine learning.

FIG. 6 shows a receiver operating characteristic curve of a model predicting the health status of patients based on records of physiological data.

FIG. 7 shows a receiver operating characteristic curve of a model comparing correct identification of healthy individuals as healthy with incorrect identification of healthy individuals as being sick.

FIG. 8 shows a scatter plot of respiration rates and resting heart rates for multiple patients.

### DETAILED DESCRIPTION

This disclosure describes a correlation between elevated heart rate, elevated respiration rate, and ill health. People who are sick, or people who are becoming sick, often have higher heart rates and higher respiration rates than healthy individuals. Heart rate and respiration rate can vary with activities such as exercise, so gradual changes over a course of multiple days may be tracked by analysis of resting heart rate and resting respiration rate. The data collected from a resting individual may be more representative of that individual's underlying health status rather than data collected during non-resting periods which may be affected by exercise, stress, or other factors. Tracking of a person's heart rate and respiration rate may provide physiological data that allows for accurate prediction that the person is becoming sick before he or she exhibits other symptoms such as a fever. Tracking of heart rate and respiration rate over time can be performed in the clinical setting when a patient is connected to various medical devices and readings of those devices are regularly recorded by nurses or other health professionals. However, for people outside of the clinical setting there are few practical ways to continuously monitor physiological data such as respiration rate and resting heart rate.

Wearable electronic devices now include sensors such as optical heart rate sensors which provide a non-invasive way of measuring heart rate and respiration rate. Optical heart rate sensors function by using an optical source to illuminate blood vessels through a user's skin and an optical sensor to measure reflected illumination from the blood vessels. Because these devices are wearable they may provide continuous, or approximately continuous, monitoring of physiological data for people outside of clinical settings.

The physiological data collected from an individual, either by a wearable device or by manual record-keeping in a clinical setting, may be analyzed by machine learning techniques to determine if the individual is likely to become sick in the near future. One type of machine learning technique that may be used is a probabilistic classification model which returns probabilities that the physiological data belongs to one or more classes representing health states such as healthy or sick. The machine learning algorithms may be trained by using data from multiple other individuals, referred to herein as “population data,” that includes heart rates, respiration rates, and the healthy or sick status of the multiple individuals. The use and training of machine learning algorithms is discussed in additional detail below. Thus, in one implementation an individual may be alerted to his or her increased likelihood of becoming sick in the near future due to elevated respiration rate and elevated heart rate measured by a wearable electronic device.

The analysis performed by machine learning techniques may be based on recently-collected physiological data such as, for example, physiological data collected over the previous few nights (e.g., data collected while the individual sleeping to obtain resting heart rate and resting respiration rate). Thus, inputs to the machine learning algorithms may include several hours of resting heart rate data and resting respiration rate data from the previous few nights. Machine learning algorithms may compare the recently-collected physiological data with the population data to determine whether the recent-collected physiological data is more like the data collected from sick individuals or more like the data collected from healthy individuals. Upon making a determination about which health status is most probable for the individual from whom the physiological data that was recently collected, a system using these machine learning techniques may report that determination. For example, in implementations in which the wearable electronic device includes a display, the system may generate a message indicating that there is a high probability of becoming sick in the near future, a medium probability of becoming sick in the near future, or a low probability of becoming sick in the near future.

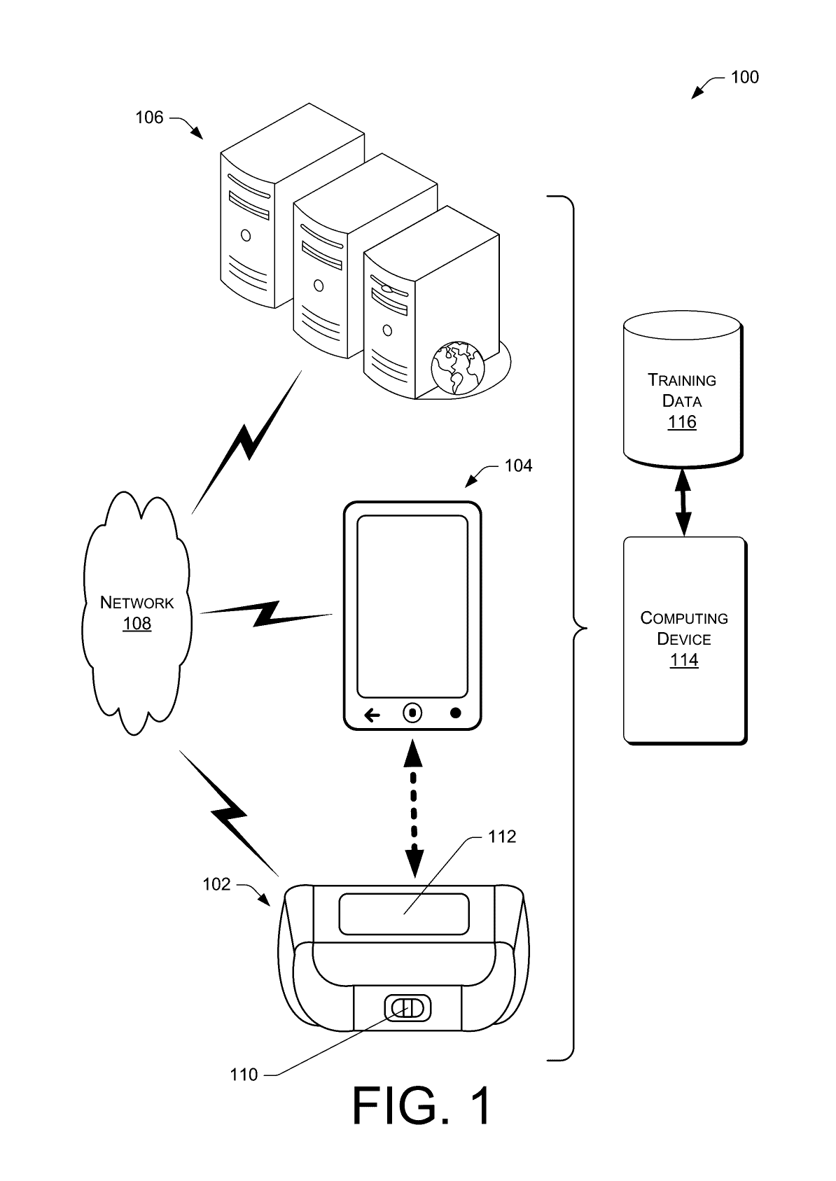

FIG. 1 shows an illustrative architecture 100 that includes a wearable electronic device 102, a mobile electronic device 104, and a computing system 106. The wearable electronic device 102 may be implemented in any number of different form factors such as jewelry, clothing, or an assistive device. Wearable electronic devices implemented as jewelry include wearable electronic devices that do not substantially cover a portion of the body and have aesthetic value but may have limited functionality other than the functionality of the wearable electronic device. Jewelry includes watches, bracelets, rings, earrings, pendants, necklaces, and the like. Wearable electronic devices implemented as clothing include wearable electronic devices that cover a portion of the body and share functionality with the analogous article of clothing that that is not a wearable electronic device. Examples of clothing include gloves, shoes, hats, headbands, wristbands, ankle bands, and the like. Wearable electronic devices implemented as an assistive device including functionality that addresses a medical need of an individual. Assistive devices include glasses, hearing aids, insulin pumps, a single-purpose device that performs monitoring of the physiological data without additional functions, and the like.

The mobile electronic device 104 is an electronic device that may be readily carried by a person. Mobile electronic devices 104 include communication devices, productivity devices, and entertainment devices. Communication devices may include mobile phones, cellular phones, pagers, email communicators, and the like. Examples of productivity devices include personal digital assistants (PDA), electronic dictionaries, digital audio recorders, and the like. Examples of entertainment devices include electronic media players, e-book readers, handhold game consoles, and the like.

The computing system 106 may be implemented as a centralized or distributed system. Centralized systems generally include one or a few separate devices containing hardware and software required for computational processing. Examples of centralized systems include desktop computers, notebook computers, tablet computers, game consoles, servers, or the like. Distributed systems generally include multiple pieces of hardware distributed across a plurality of locations. For example, a server farm containing many different servers interacting together is a distributed system. A cloud computing system that may use processing power, memory, and other hardware resources from multiple different geographic locations is also an example of a distributed system.

Each of the wearable electronic device 102, the mobile electronic device 104, and a computing system 106 may be in communication with a network 108. The network 108 may be implemented as any type of communications network such as a local area network, a wide area network, a mesh network, and ad hoc network, a peer-to-peer network, the Internet, a cable network, a telephone network, and the like. The wearable electronic device 102, the mobile electronic device 104, and a computing system 106 may be directly connected to the network 108 or indirectly connected to the network 108 via one or more other types of communication infrastructure. For example, the wearable electronic device 102 may be directly connected to a wireless cellular telephone network that is in turn connected to the network 108.

Additionally, the wearable electronic device 102 may have a communicative connection to the mobile electronic device 104 that does not go through the network 108. This connection may be a direct connection or may be mediated by one or more other devices. The communicative connection between the wearable electronic device 102 and the mobile electronic device 104 may be implemented as a wired connection or a wireless connection. The wired connection may include one or more wires or cables physically connecting the wearable electronic device 102 to the mobile electronic device 104. For example, the wired connection may be created by a headphone cable, a USB cable, an Ethernet cable, or the like. The wireless connection may be created by radio frequency (e.g., any version of Bluetooth, ANT, Wi-Fi IEEE 802.11, etc.), infrared light, or the like.

The wearable electronic device 102 may include multiple input and output devices such as one or more physiological sensor(s) 110 and a display screen 112. The physiological sensor(s) 110 may detect both heart rate and respiration rate. One technique for detecting both heart rate and respiration rate is a photoplethysmogram (PPG) which is an optically obtained volumetric measurement of an organ. To obtain a PPG the physiological sensor(s) 110 may be implemented as an optical sensor which includes a light source (e.g. a light emitting diode) configured to illuminate a wearer's skin and a matched optical sensor (e.g., a photodiode) configured to detect light at frequencies that are based on the frequencies of light output by the light source. The optical sensor can measure heart rate by measuring the amount of light reflected back from a wearer's skin to the optical sensor. With each heartbeat blood surges the arteries and veins and then ebbs. Blood absorbs more light than surrounding tissue so differences in the amount of blood result in differences in the absorbance of light. This change in absorbance of light can be detected by the optical sensor and correlated with a heart rate. The same optical sensor can also detect respiration rate because blood flow is effected by movement of the lungs as well as the heart. Respiration effects the cardiac cycle by varying the intrapleural pressure, the pressure between the thoracic wall and the lungs. Since the heart resides in the thoracic cavity between the lungs, the partial pressure of inhaling and exhaling greatly influence the pressure on the vena cava and the filling of the right atrium. This in turn affects the volume of blood flow in cycle with respiration. The change in blood flow leads to a change in absorbance of light which again can be detected by the optical sensor.

Other physiological sensor(s) 110 that are capable of detecting heart rate include sensors that detect electrical signals generated by the heart such as electrodes placed on a person's body. Examples of physiological sensor(s) 110 that are capable of detecting respiration rate include chest or abdomen straps that detect chest expansion, electrodes that measure electrical impedance across a wearer's thorax. During inhalation, there is an increase in the gas volume of the chest in relation to the fluid volume; this increase causes conductivity to decrease. During inhalation, the length of the conductance paths increases because of expansion. The increase in gas volume and expansion of the chest both result in variations in impedance which can be correlated with respiration rate. Acoustic monitoring through a stethoscope or a wearable acoustic sensor can detect acoustical signals produced by airflow during inhalation and exhalation. Thus, an acoustic monitor is a further example of a physiological sensor(s) 110 that can detect a respiration rate. In some implementations, the wearable electronic device 102 may include a first physiological sensor for measuring heart rate and a second, different physiological sensor for measuring respiration rate.

The wearable electronic device 102 may also include additional physiological sensors (not shown) such as a skin temperature sensor, a galvanic skin response (GSR) sensor, a blood pressure sensor, and the like. The wearable electronic device 102 may additionally include sensors that are used to detect non-physiological data; such sensors may include a gyroscope, a light sensor, and the like, although these sensors may be used to determine physiological data either in addition or instead. The wearable electronic device 102 may additionally include other components (not shown) such as a power source (e.g., battery), radio transmitter, radio receiver, and the like.

Display screen 112 on the wearable electronic device 102 may output information such as readings from the physiological sensor(s) 110 and/or other sensors on the wearable electronic device 102. The wearable electronic device 102 may include other output devices (not shown) such as speakers or an audio output jack. In some implementations output may be generated through audio instead of or in addition to visual output.

Architecture 100 shows a computing device 114 that may be implemented in whole or in part by any combination of the wearable electronic device 102, the mobile electronic device 104, and/or the computing system 106. Thus, in one implementation the wearable electronic device 102 functions as a standalone computing device 114 that detects physiological data, implements machine learning algorithms, and characterizes the physiological data as corresponding to a probability that a person will become sick and/or develop a fever. In one implementation, the wearable electronic device 102 collects the raw data from the physiological sensor(s) 110 and all subsequent analysis of the physiological data is performed by the computing system 106. In one implementation, the wearable electronic device 102 collects data from the physiological sensor(s) 110 and all subsequent analysis of the physiological data is performed by the mobile electronic device 104. In one implementation, the wearable electronic device 102 collects physiological data from the physiological sensor(s) 110, the physiological data is passed to the mobile electronic device 104 for processing, and the computing system 106 provides additional processing and archiving of the physiological data. Other implementations and distribution of the functionalities between the wearable electronic device 102, the mobile electronic device 104, and the computing system 106 are also possible. Thus, the computing device 114 as described herein may include implementations of this disclosure that are performed by one or more of the wearable electronic device 102, the mobile electronic device 104, and or the computing system 106.

The computing device 114 additionally has access to training data 116 for use by machine learning techniques to train algorithms that analyze the physiological data generated by the physiological sensor(s) 110. The training data 116 may include data from a population of individuals (“population data”) who are different from the individual wearing the wearable electronic device 102. The training data 116 may also be past physiological data (“historical data”) collected from the individual wearing the wearable electronic device 102. The historical data may be physiological data collected from the physiological sensor(s) 110 or it may be formed in whole or in part from physiological data collected by sensors that are not associated with the wearable electronic device 102. The training data 116 may include a series of records each having a heart rate and a respiration rate value. This series of records may include additional information as well. The records may include time. For example, one record may include information such as: Oct. 29, 2014, 11:37 PM, heart rate=87, respiration rate=22. The training data 116 may be stored in whole or in part on either of the wearable electronic device 102, the mobile electronic device 104, and/or the computing system 106. The training data 116 may also be stored on a separate device (not shown) and accessed by the computing device 114 during training of machine learning algorithms. Thus, computing device 114 may not use the training data 116 except to provide a training set for the machine learning which creates the specific algorithms implemented by the computing device 114.

### Illustrative Computing Device

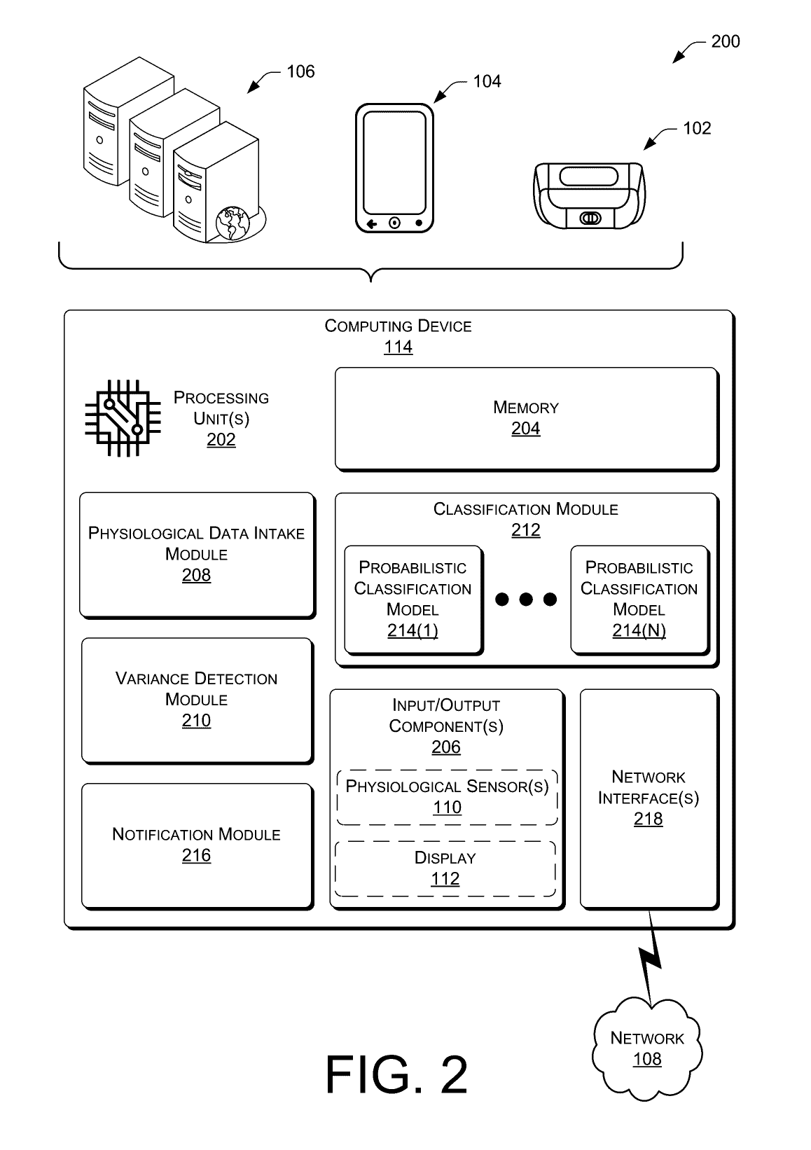

FIG. 2 shows an illustrative block diagram 200 of components that may be included in the computing device 114 of FIG. 1. The computing device 114 may contain one or more processing unit(s) 202 and memory 204 both of which may be distributed across one or more physical or logical locations. The processing unit(s) 202 may include any combination of central processing units (CPUs), graphical processing units (GPUs), single core processors, multi-core processors, application-specific integrated circuits (ASICs), programmable circuits such as Field Programmable Gate Arrays (FPGA), and the like. One or more of the processing unit(s) 202 may be implemented in software and/or firmware in addition to hardware implementations. Software or firmware implementations of the processing unit(s) 202 may include computer- or machine-executable instructions written in any suitable programming language to perform the various functions described. Software implementations of the processing unit(s) 202 may be stored in whole or part in the memory 204.

The memory 204 may include removable storage, non-removable storage, local storage, and/or remote storage to provide storage of computer readable instructions, data structures, program modules, and other data. The memory 204 may be implemented as computer-readable media. Computer-readable media includes, at least, two types of media, namely computer-readable storage media and communications media. Computer-readable storage media includes volatile and non-volatile, removable and non-removable media implemented in any method or technology for storage of information such as computer-readable instructions, data structures, program modules, or other data. Computer-readable storage media includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other non-transmission medium that can be used to store information for access by a computing device.

In contrast, communications media may embody computer readable instructions, data structures, program modules, or other data in a modulated data signal, such as a carrier wave, or other transmission mechanism. As defined herein, computer-readable storage media and communications media are mutually exclusive.

The block diagram 200 shows multiple modules included within the computing device 114. These modules may be implemented in software and alternatively, or additionally, implemented, in whole or in part, by one or more hardware logic components or firmware. For example, and without limitation, illustrative types of hardware logic components that can be used include Field-programmable Gate Arrays (FPGAs), Application-specific Integrated Circuits (ASICs), Application-specific Standard Products (ASSPs), System-on-a-chip systems (SOCs), Complex Programmable Logic Devices (CPLDs), etc. Each of the modules may be implemented in the same manner (e.g., all software) or individual ones of the modules may have separate implementations (e.g., some as software, others as ASICs, and yet others as CLPDs).

The computing device 114 may include one or more input/output components(s) 206 such as a keyboard, a pointing device, a touchscreen, a microphone, a camera, a display, a speaker, a printer, and the like. Implementations of the computing device 114 that include the wearable electronic device 102 may include the physiological sensor(s) 110 as an input component and may include the display 112 as an output component.

A physiological data intake module 208 may receive data from the physiological sensor(s) 110. The physiological data intake module 208 may receive physiological data with or without accompanying time data. If the physiological data is received without accompanying time data, the physiological data intake module 208 may add a timestamp to the physiological data. In implementations in which the physiological data intake module 208 is located in a different device than the physiological sensor(s) 110, the physiological data intake module 208 may be configured to receive physiological data from the one or more physiological sensors via one or more network interfaces such as the connection between the wearable electronic device 102 and the network 108 or the communicative connection between the wearable electronic device 102 and the mobile electronic device 104.

After receiving the physiological data, the physiological data intake module 208 may cause the computing device 114 to store the physiological data in the memory 204. The physiological data may be stored in association with one or more physiological data descriptors. For example, heart rate data may be stored in association with a data descriptor for heart rate. Similarly, respiration rate data may be stored association with a data descriptor for respiration rate. For example, the value 60 may represent either a respiration rate or a heart rate. Storing a raw value in association with a data descriptor allows for proper identification and processing of the stored value. The physiological data intake module 208 may also include filtering functionality that receives but does not store data that appears to be erroneous. This may be implemented by discarding data which is above a maximum threshold or below a minimum threshold for particular type of data. For example, a negative heart rate or negative respiration rate may be discarded. Similarly a heart rate or respiration rate above 500 beats-per-minute (bpm) or respirations per minute may also be discarded. Erroneous data may be introduced in many ways such as in errors created by the physiological sensor(s) 110, errors in transmission, or other ways.

A variance detection module 210 may detect variations between the physiological data received by the physiological data intake module 208 and one or more baseline values. The baseline values may represent values for physiological data that are typical or ordinary for a wearer of the wearable electronic device 102. For example, baseline values may be determined by analysis of the historical data from a given individual. Each baseline value may be associated with a physiological data descriptor such as heart rate or respiration rate. In an implementation, baseline heart rate, or other physiological data descriptor, may be a representative value for the resting heart rate of the individual collected over a previous period of time. The previous period of time may be the previous two to 90 days. In an implementation the previous period of time may be the previous three to 14 days. In an implementation the previous period of time may be the previous five to 10 days. In an implementation the previous period of time may be the previous seven days. The representative value may be a mean, median, or mode. Of course the baseline value may be derived from data collected over more than or fewer than seven days. In one implementation the variance detection module 210 may compare physiological data stored in the memory 204 with one or more baseline values for individual ones of the physiological data descriptors. Thus, for example, physiological data stored in association with the physiological data descriptor for respiration rate may be compared with a baseline respiration rate for the individual from whom the physiological data was collected.

Physiological data may be identified as “resting” data when that data is collected at a time that the individual is determined to be at rest or sleep. The status of individual as resting could be indicated manually by the individual whenever he or she prepares to sleep. The end of resting could also be indicated manually when the individual wakes. Resting status may be detected automatically by a wearable electronic device, such as the wearable electronic device 102 shown in FIG. 1). The wearable electronic device may use any number of different sensors to detect or and for that the individual is resting. For example a motion sensor or gyroscope in the wearable electronic device may detect motion of the individual and the absence of motion may be used to infer that the individual is resting. Measurement of physiological features such as skin temperature, heart rate, and or respiration rate may also be used to infer a state of rest. It is known that skin temperature, heart rate, and respiration rate all decrease during normal sleep. Thus, measurement of any of these metrics over time may be used to identify the individuals sleep and wake cycle. A wearable electronic device that detects brain waves such as an electroencephalogram (EEG) may detect that the individual is sleeping. The status of the individual as resting may also be inferred based on time. For example, the individual may be assumed to be resting or asleep between the times of 1 AM and 5 AM.

The baseline values for physiological data descriptors may be stored as part of a user profile which can additionally include information such as username, globally unique user identifier, gender, age, weight, height, and the like. The user profile may be stored in the memory 204. Thus, the variance detection module 210 may receive physiological data from the physiological data intake module 208, access stored baselines in the memory 204, and determine if and how much the physiological data associated with a given physiological data descriptor varies from the baseline for that same physiological data descriptor.

The variance detection module 210 may determine if a given physiological datum varies from the corresponding baseline value by more than a threshold amount. The threshold amount may be a fixed amount (e.g., beats per minute for heart rate, 15 breaths per minute for respiration rate, etc.) or a variable amount that depends on the value of the baseline (e.g., 5%, 10%, 15%, 25%, etc. of the baseline). Thus, the variance detection module 210 may flag physiological data that is “abnormal” in that it differs from the baseline value for a given physiological data descriptor. The flag implemented by any suitable technique such as appending metadata to a record which contains physiological data which varies from the baseline by more than a threshold amount. In an implementation, the flag may be implemented by storing the physiological data separately in the memory 204. In an implementation, the flag may additionally or alternatively be implemented by only passing physiological data that varies from the baseline by more than a threshold amount to a separate module or separate device.

A classification module 212 may receive physiological data and classify the physiological data as likely representing a given health status such as healthy or sick. The classification module 212 may receive physiological data and also receive associated physiological data descriptors from the memory 204. In an implementation the classification module 212 may receive physiological data from the physiological data intake module 208 in response to the variance detection module 210 detecting a variation from a baseline value. The classification module 212 may analyze all available physiological data for it may analyze a subset of the available physiological data. For example, the classification module 212 may analyze physiological data from the previous 24 hours, 48 hours, 72 hours, 96 hours, or another length of time.

In implementations in which the classification module 212 analyzes the physiological data in response to the variance detection module 210 detecting a variance, the classification module 212 may not analyze physiological data when the physiological data varies less than the threshold amount from the appropriate baseline. Thus, in such implementations the variance detection module 210 may act as a trigger to determine when the classification module 212 acts to analyze physiological data. Thus, operation of the variance detection module 210 may reduce instances of operation of the classification module 212 which can provide savings in processor load, energy usage, and network communications. For example, in an implementation in which the variance detection module 210 is implemented in the wearable electronic device 102 and the classification module 212 is implemented in the computer system 106, the wearable electronic device 102 would contact the computing system 106 to request analysis of physiological data when the physiological data has sufficient variance from the baseline. This reduces frequency of communication between the mobile electronic device 102 and the computing system 106. Additionally, shifting the functionality of the classification module 212 to the computing system 106 reduces processor load and energy consumption of the mobile electronic device 102.

The classification module 212 may classify physiological data (e.g., received from the memory 204) by use of one or more probabilistic classification models 214(1) . . . 214(N). The probabilistic classification models 214 are generated by machine learning techniques that construct algorithms from example inputs and are able to use the construct algorithms to make predictions, decisions, or classifications from new information. Probabilistic classification is a type of machine learning that given a sample input is able to predict a probability distribution over set of classes. For example, given a patient's heart rate and respiration rate a probabilistic classifier can determine a probability that the patient is healthy and a probability that the patient is sick. Probabilistic classification models can include mixture models, discriminant analysis models, and discriminative models. Probabilistic classification includes supervised learning which is the machine learning task of inferring a function from labeled training data. The labeled training data may be the training data 116 from FIG. 1. The training data consist of a set of training examples. In supervised learning, each example is a pair consisting of an input object (typically a vector) and a desired output value (also called the supervisory signal). A supervised learning algorithm analyzes the training data and produces an inferred function, which can be used for mapping new examples. An optimal scenario will allow for the algorithm to correctly determine the class labels for unseen instances. This requires the learning algorithm to generalize from the training data to unseen situations in a “reasonable” way.

Mixture models are probabilistic models for representing the presence of subpopulations within an overall population, without requiring that an observed data set should identify the sub-population to which an individual observation belongs. Formally a mixture model corresponds to the mixture distribution that represents the probability distribution of observations in the overall population. A typical finite-dimensional mixture model is a hierarchical model consisting of the following components: N random variables corresponding to observations, each assumed to be distributed according to a mixture of K components, with each component belonging to the same parametric family of distributions (e.g., all Normal, all Zipfian, etc.) but with different parameters; N corresponding random latent variables specifying the identity of the mixture component of each observation, each distributed according to a K-dimensional categorical distribution; a set of K mixture weights, each of which is a probability (a real number between 0 and 1 inclusive), all of which sum to 1; and a set of K parameters, each specifying the parameter of the corresponding mixture component. In many cases, each “parameter” is actually a set of parameters. For example, observations distributed according to a mixture of one-dimensional Gaussian distributions will have a mean and variance for each component. Observations distributed according to a mixture of V-dimensional categorical distributions (e.g., when each observation is a word from a vocabulary of size V) will have a vector of V probabilities, collectively summing to 1. Examples of mixture models include Gaussian mixture models (GMM), multivariate Gaussian mixture models, and categorical mixture models. Each of these types of models is well understood by those having ordinary skill in the art.

Discriminant analysis is a statistical analysis to predict a categorical dependent variable (called a grouping variable) by one or more continuous or binary independent variables (called predictor variables). Discriminant analysis is used when groups are known a priori. Each case must have a score on one or more quantitative predictor measures, and a score on a group measure. In simple terms, discriminant function analysis is classification—the act of distributing things into groups, classes or categories of the same type. Discriminant analysis works by creating one or more linear combinations of predictors, creating a new latent variable for each function. These functions are called discriminant functions. The number of functions possible is either Ng−1 where Ng=number of groups, or p (the number of predictors), whichever is smaller. The first function created maximizes the differences between groups on that function. The second function maximizes differences on that function, but also must not be correlated with the previous function. This continues with subsequent functions with the requirement that the new function not be correlated with any of the previous functions. One discriminant analysis model is linear discriminant analysis (LDA) which finds a linear combination of features that characterizes or separates two or more classes of objects or events.

The resulting combination may be used as a linear classifier or for dimensionality reduction before later classification. Other discriminant analysis models include analysis of variance (ANOVA) and multivariate analysis of variance/multiple analysis of variance (MANOVA). Other discriminant analysis models include principal component analysis (PCA) and factor analysis. Each of these types of models is well understood by those having ordinary skill in the art.

Discriminative models use an object's characteristics to identify which class (or group) it belongs to. A discriminative model achieves this by making a classification decision based on the value of a linear combination of the characteristics. An object's characteristics are also known as feature values and are typically presented to the machine in a vector called a feature vector. Discriminative models attempt to maximize the quality of the output on a training set. Additional terms in the training cost function can perform regularization of the final model. Discriminative models are used in machine learning for modeling the dependence of an unobserved variable y on an observed variable x. Within a probabilistic framework, this is done by modeling the conditional probability distribution P(y|x), which can be used for predicting y from x. Examples of discriminative models include logistic regression, support vector machines (SVM), boosting, conditional random fields, linear regression, and neural networks. SVMs, for example, are supervised learning models with associated learning algorithms that analyze data and recognize patterns, used for classification and regression analysis. Given a set of training examples, each marked as belonging to one of two categories, an SVM training algorithm builds a model that assigns new examples into one category or the other, making it a non-probabilistic binary linear classifier. Each of these types of models is well understood by those having ordinary skill in the art.

Upon classifying the physiological data with one or more probabilistic classification model(s) 214 the classification module 212 may return probabilities that the physiological data belongs to one or more classes representing health states (e.g. healthy, ambiguous, or sick). In an implementation the classification module 212 may select the health state having a highest probability as compared to the other health states as the classification for a given set of results from the physiological data. The classification module 212 may also select the health state by other techniques. This classification may be passed on or reported to another module such as a notification module 216.

The notification module 216 may send the classification to another device or to an output component 206. In an implementation in which the notification module 216 is located on the computing system 106, the notification module 216 may send the resulting classification to the mobile electronic device 104 and/or the wearable electronic device 102. Classification may then be output by an output device on the mobile electronic device 104 and/or the wearable electronic device 102. In an implementation in which the notification module 216 is located in the wearable electronic device 102, the resulting classification may be output on the display 112. The classification may reach the wearable electronic device via one or more network interfaces 218 of the wearable electronic device 102 via the network 108. The network interface(s) 218 include hardware and/or software for enabling any or all of the computing system 116, the mobile electronic device 104, and the wearable electronic device 102 to access and communicate via the network 108.

In an implementation the notification module 216 may cause a display on the mobile electronic device 104 and/or the display 112 on the wearable electronic device 102 to display a color and or a symbol associated with the health state having a highest probability as determined by the one or more probabilistic classification model(s) 214. For example, physiological data that is classified as indicating sickness may be represented by the color red, physiological data that classified indicate health may be represented by the color green, and physiological data that is ambiguous or difficult to clearly classified as either corresponding with health or sickness may be represented by the color yellow. Of course alternate representations part also possible such as textual descriptions such as “sick,” “healthy,” and “ambiguous,” or “possibly becoming sick.” Similarly, an output device other than a display may be used to provide the resulting classification to an end-user. For example, audio output may generate different tones depending on the health state classification, a voice may be generated which verbally tells the end-user of the classified health state.

In an implementation the notification module 216 may additionally or alternatively report the health state classification to an electronic device that is not associated with the person from whom the physiological data was collected. For example, upon receiving appropriate permission from the person from whom the physiological data was collected, the notification module 216 may securely send the classification to an electronic device associated with another individual or organization such as a caregiver like a nurse, physician, hospital, or the like.

### Illustrative Processes

For ease of understanding, the processes discussed in this disclosure are delineated as separate operations represented as independent blocks. However, these separately delineated operations should not be construed as necessarily order dependent in their performance. The order in which the process is described is not intended to be construed as a limitation, and any number of the described process blocks may be combined in any order to implement the process, or an alternate process. Moreover, it is also possible that one or more of the provided operations may be modified or omitted.

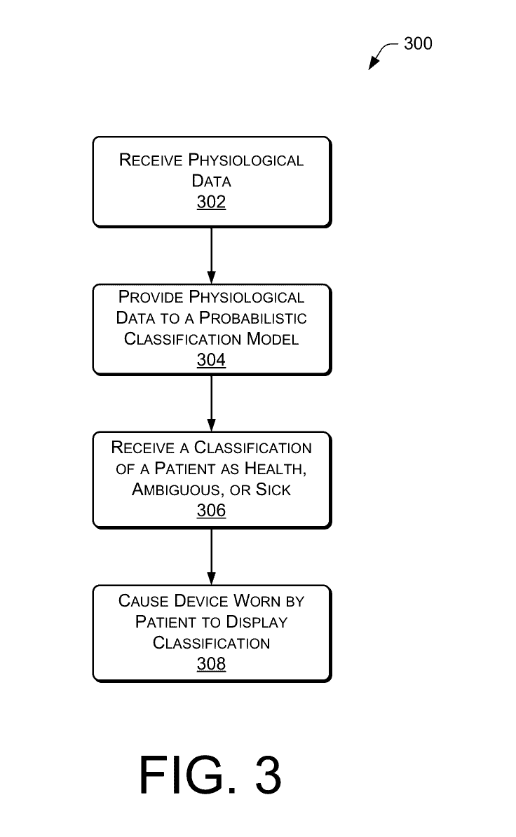

FIG. 3 shows process 300 which classifies a patient's health status as healthy, ambiguous, or sick. The process 300 may be implemented by the computing device 114 shown in FIGS. 1 and 2.

At 302, physiological data is received from a patient. The physiological data may include heart rate and/or respiration rate. In an implementation the physiological data may include resting heart rate and/or resting respiration rate. The physiological data may be received from any type of sensors capable of detecting the physiological data. In an implementation the physiological data may be received from an optical sensor worn by the patient. The optical sensor may be part of a conventional medical sensor implemented in a clinical setting or the optical sensor may be part of a device worn by the patient such as the wearable electronic device 102. In an implementation the optical sensor may be physiological sensor 110 shown in FIG. 1.

At 304, the physiological data is provided to a probabilistic classification model such as the probabilistic classification model 214 shown in FIG. 2. The probabilistic classification model may be created by a machine learning technique. The machine learning technique may include supervised learning based on physiological data used as training data such as training data 116. The physiological data used as training data may be population data from a plurality of individuals classified as healthy and a plurality of individuals classified as sick. The physiological data used as training data may also be historical data collected from past records of the patient. The past records of the patient may be labeled as representing times when the patient as healthy or as times when the patient was sick. The labeling may be performed manually by the patient or automatically such as associating a particular physiological condition with sickness. For example, any time the core temperature of the patient exceeded 100° F. (37.8° C.) those records may be labeled as representing sickness. The probabilistic classification model may use any appropriate model such as a mixture model, a discriminant analysis model, or a discriminative model.

At 306, a classification of the health state of the patient is received from the probabilistic classification module. The classification may be a health state selected from the health states of healthy, ambiguous, or sick. The classification may additionally or alternatively include a first probability that the patient's health status is correctly classified as healthy and a second probability that the patient's health status is correctly classified as sick. The classification may be based on the first probability and the second probability as well as potentially other information.

As an example, the probabilistic classification model may return a 57% probability that the health status is healthy and a 15% probability that the health status is sick. In an implementation the highest probability health status is selected, so for this example the patient's health status would be classified as healthy. As an additional example, the probabilistic classification model may return a 42% probability that the health status is healthy and a 39% probability that the health status is sick. In an implementation when the two probabilities are within a threshold range of each other (e.g., 5%, 10%, 15%, 20%, etc.) the probabilistic classification model may classify the patient's health status as ambiguous because it is difficult for the model to determine whether a healthy or a sick classification is most likely to be correct. As an additional example, the probabilistic classification model may return a 12% probability that the health status is healthy and a 1% probability that the health status is sick. Although healthy is the most probable classification and healthy is 10% more likely than a sick classification, in an implementation the probabilistic classification model may classify the patient's health status as ambiguous if neither the healthy probability nor the sick probability are above a threshold probability level (e.g., 20%, 30%, 40%, 50%, 60%, 70%, etc.). Other configurations and thresholds for interpreting the probabilities are also possible.

At 308, a device worn by the patient may display the classification of the patient as healthy, ambiguous, or sick. In an implementation the device may be the wearable electronic device 102 of FIG. 1. The device may display the words “healthy,” “ambiguous,” or “sick” depending upon the corresponding classification. The device may alternatively or additionally display a color and/or symbol corresponding to the classification.

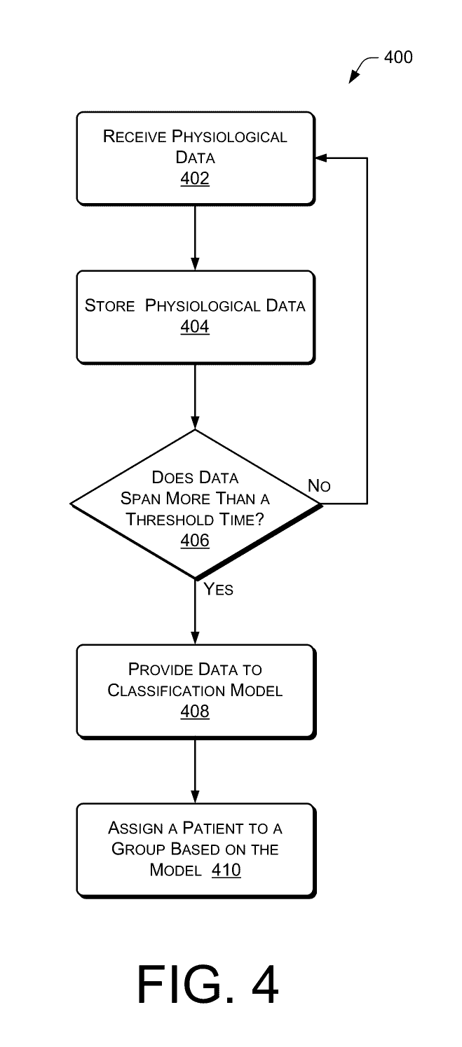

FIG. 4 shows process 400 which uses the classification model to determine if physiological data suggest that a patient is likely or unlikely to develop a fever. The process 400 may be implemented by the computing device 114 shown in FIGS. 1 and 2.

At 402, physiological data is received from a patient. The physiological data may include any type of physiological data such as, but not limited to respiration rate data, heart rate data, or both. The physiological data may be generated by a wearable electronic device on the patient such as, for example, the wearable electronic device 102 shown in FIG. 1.

At 404, a plurality of time points of the physiological data may be stored. Each time point may include a time together with physiological data. For example, a time point may include a date and time, a heart rate, and a respiration rate. Data collected over time can show trends. The time points of the physiological data may be stored in memory 204 shown in FIG. 2.

At 406, is determined if the plurality of time points of the physiological data stored at 404 stands at least a first threshold length of time. If the physical data has been collected over a sufficiently long (i.e., first threshold length) period of time, the physiological data may be useful for determining trends. The first threshold length of time may be any length of time sufficient for collecting data to support further analysis. Thus, the threshold length of time may vary depending on what subsequent analysis will be performed in the plurality of time points of physiological data. In some implementation, the first threshold length of time may be any time between about 12 and about 120 hours. In some implementations, the first threshold length of time may be 12, 24, 36, 48, 72, 96, or 120 hours. In one invitation, the first threshold length of time may be 72 hours.

If the plurality of time points of the physiological data does not span the first threshold length of time, then process 400 returns to 402 and receives additional physiological data which in turn is stored at 404 until there is a long enough span of physiological data available. If the plurality of time points of the physiological data spans the first threshold length of time, then process 400 proceeds to 408.

At 408, the plurality of time points of physiological data is provided to a probabilistic classification model. The probabilistic classification model may be the probabilistic classification model 214 shown in FIG. 2. The probabilistic classification model is created by supervised machine learning from a set of training data. The set of training data may be the training data 116 shown in FIG. 1. In an implementation the set of training data may include physiological data from a plurality of individuals within the patient who are classified as healthy and a plurality of individuals other than the patient who are classified as sick. In an implementation the set of training data may include data previously collected from the patient such as the patient's historical data.

The set of training data may additionally include core temperature readings from the plurality of individuals. The core temperature readings a be measured by any technique that directly or indirectly measures core body temperature such as, but not limited to, skin temperature sensors, in-ear thermometers, oral thermometers, rectal thermometers, thermography, and the like. The probabilistic classification model may use the training data to identify physiological data which is predictive of a patient later developing a fever. For the purposes of the probabilistic classification models, a fever may be classified as a core body temperature above a threshold temperature. In one implementation the threshold temperature for classifying a core body temperature as representing a “fever” is 100° F. (37.8° C.). Thus, for example, following training with an appropriate set of training data, the probabilistic classification model may generate a probability that physiological data other than core temperature (e.g., heart rate and or respiration rate) predicts a future increase in core temperature to a level that is classified as a “fever.”

Upon providing the plurality of time points of physiological data to the probabilistic classification model, the probabilistic classification model returns probabilities that the patient belongs to one of a number of different groups. In an implementation, the groups comprise two groups: a group that will develop a fever within a second threshold length of time and a group that will not develop a fever within the second threshold length of time. In an implementation, the groups comprise three groups, a group that will develop a fever within a second threshold length of time, a group that will not develop a fever within the second threshold length of time, and a group that may or may not develop a fever within the second threshold length of time. In other words, the probabilistic classification model classify the patient as someone who will get a fever or someone will not get a fever, or alternatively as someone who will get a fever, someone will not get a fever, or someone who might get a fever. In one implementation, someone who will not get a fever may be equated to the classification of “healthy,” someone who will get a fever in the future may be equated to the classification of “sick,” and someone who might get a fever in the future may be equated to the classification of “ambiguous.” Of course, classification into other groups is also possible such as, for example, a group that will develop a high fever (e.g., above 103° F. (39.4° C.)) and a group that will develop a low fever (e.g., between 100° F. and 103° F. (37-39.4° C.)). The second threshold length of time may represent some period of time in the near future or the idea of “soon.” In an implementation, the second threshold length of time is between about 12 hours and about 72 hours. In an implementation, the second threshold length of time is 12, 18, 24, 30, 36, 42, 48, 54, 60, 66, or 72 hours. In one implementation, the second threshold length of time is 24 hours. In some implementations, the second threshold length of time may be equal to or shorter than the first threshold length of time, equal to or shorter than one half of the first threshold length of time, or equal to or shorter than one third of the first threshold length of time.

At 410, the patient is assigned to one of the groups based on the probabilities returned from the probabilistic classification model. In an implementation, the patient may be assigned to the group associated with the highest probability. In an example, if the probabilistic classification model returns a 70% probability of the patient developing a fever within the next 24 hours and a 20% probability of the patient not developing a fever within the next 24 hours, then the patient may be assigned to the group that will develop a fever within the threshold length of time. In an example, if the probabilistic classification model returns a 30% probability that the patient will develop a fever within the next 36 hours and a 30% probability that the patient will not develop a fever within the next 36 hours, then the patient may be assigned to the group that may or may not develop a fever within the next 36 hours.

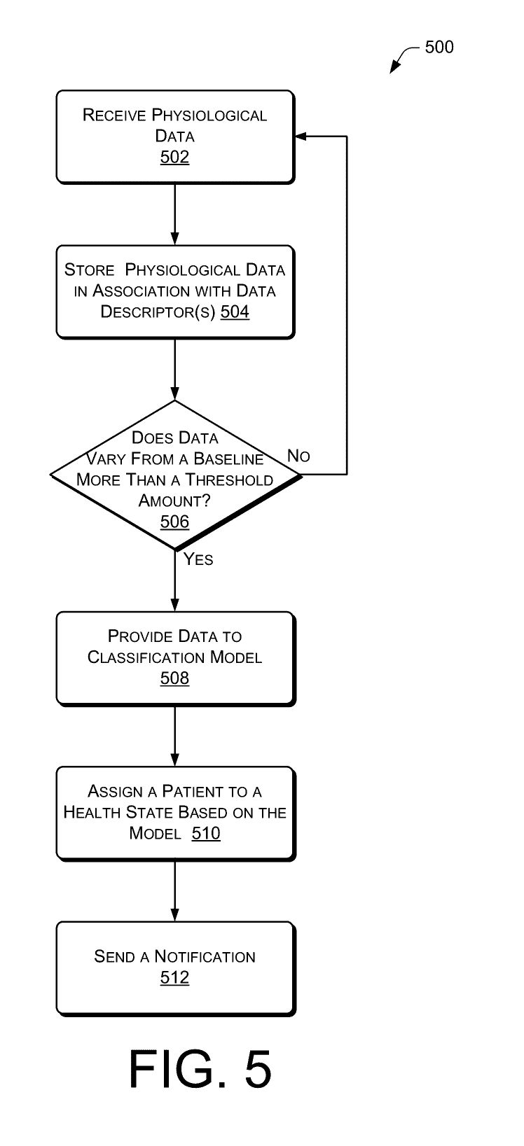

FIG. 5 shows process 500 which uses a classification model to determine which health state is the most probable classification for a patient based on based on that patient's physiological data. The process 500 may be implemented by the computing device 114 shown in FIGS. 1 and 2.

At 502, physiological data of the patient is received. The physiological data may be received from a data store (e.g., previously reported data) or the physiological data may be received directly from physiological sensor(s). In one implementation the physiological sensor(s) may be an optical sensor, and the physiological data may be heart rate data and respiratory rate data both collected by the optical sensor. The physiological data may be smoothed using a median filter or other technique to remove or reduce the influence of outlying data. The median filter keeps a media value for each set of data of a given type of physiological data over a given time period. Values other than the median are not used in subsequent analysis. The given period of time may be, for example, a 6-hour period, a 12-hour period, an 18-hour period, a 24-hour period, or another length of time. In an implementation the physiological data may be smoothed by taking the average value for a given type of physiological data over the given time period.

At 504, the physiological data may be stored in memory in association with one or more data descriptor(s). The memory may be memory 204 shown in FIG. 2. The physiological data descriptor(s) may be provided by the physiological sensor(s), a device which includes the physiological sensor(s) (e.g., wearable electronic device 102), or a device which stores the physiological data (e.g., computing device 114). The data descriptor(s) may be associated with the physiological data as column or category headings in a database record. The data descriptor(s) may indicate the physiological property that was measured by a given datum from the physiological data (e.g., heart rate, respiration rate, etc.).

At 506, is determined if the physiological data stored in the memory varies from one or more a baseline values by more than a threshold amount. It may be determined if physiological data is within the range of “normal” data or if the physiological data represents “atypical” data. For a given data descriptor the baseline may be developed by taking a representative value of the corresponding physiological data. For example, an average is one type of representative value. The threshold may be based on a percentage of the representative value. The threshold may also account for variance in the physiological data by measuring standard deviation. For example, a threshold may be +/−10% of the representative value or +/−one standard deviation of the representative value. Physiological data that varies from the baseline by less than the threshold amount may be interpreted as “normal” data. Physiological data that varies from the baseline by more than the threshold amount may be interpreted as “atypical” data. Each physiological data descriptor may be associated with a unique baseline and with a unique threshold amount. For example, the baseline for heart rate may be a different value than the baseline for respiration rate. Similarly, the threshold amount of variation that is considered normal for heart rate may be a different amount than the threshold amount of variation that is considered normal for respiration rate.

If the physiological data does not vary from the baseline by more than a threshold amount, process 500 returns to 502 where additional physiological data is received. Thus, process 500 may continue in a loop until physiological data is received which varies from the baseline by more than a threshold amount. If the physiological data varies from the baseline by more than a threshold amount, process 500 proceeds to 508.

At 508, the physiological data is provided to a probabilistic classification model that returns probabilities that the physiological data belongs to one or more classes representing different health states. The probabilistic classification model may be a mixture model, a discriminant analysis model, or a discriminative model. The probabilistic classification model may be the probabilistic classification model 214 shown in FIG. 2. In an implementation, the classes representing health states may be healthy, ambiguous, and sick. In an implementation, the classes representing health states may be likely to develop a fever, unclear if a fever will or will not develop, and unlikely to develop a fever.

At 510, the patient is assigned to a health state that has a highest probability of being the correct characterization of the patient's physiological data as determined by the probabilistic classification model.

At 512, notification of the health state which was assigned to the patient is sent to a recipient device. In an implantation the recipient device may be the wearable electronic device 102. The recipient device may also be the mobile electronic device 104. In some implementations, the recipient device may be a device that is not associated with the patient such as a computing device of a caregiver, nurse, position etc. that may receive a text message or email message with the health state. The notification of the health state may also be sent to multiple recipient devices.

### Training Data and Testing

Physiological data was collected from a set of hospitalized patients and non-hospitalized, healthy individuals. Nurses recorded heart rate, respiration rate, blood pressure, core temperature (measured in-ear), and peripheral capillary oxygen saturation (SpO₂) from a group of 66 hospitalized individuals two or three times each day over a course of a hospital stay which typically ranged from two to ten days but was longer from some patients. This generated 562 individual records. Each individual record includes collection time, heart rate, respiration rate, blood pressure, temperature, and SpO₂. Out of the 66 hospitalized individuals, 52 were each provided with a Microsoft Band equipped with an optical heart rate sensor configured to detect both heart rate and respiration rate. This generated 475 records. The records generated by the Microsoft Bands include time, heart rate, and respiration rate. Additionally, physiological data was collected from a set of 25 non-hospitalized individuals considered to be healthy. This generated 47 records. Each of the records of the healthy individuals included heart rate, respiration rate, blood pressure, temperature, and SpO₂.

Each individual record collected by the nurses was manually labeled to create a set of training examples for use in machine learning. Microsoft Bands were used to collect data from the hospitalized patients in duplicate with data collected by the nurses. A physician reviewed the data collected by the nurses and for each record characterized the patient's health state as sick, ambiguous, or healthy. Record keeping began for the 66 people included in the patient population while those individuals were in the hospital, so those individuals that presented “healthy” physiological data generally did so as a result of recovering from the ailment that necessitated the hospital stay. Thus, the characterization of physiological data for the patients in the hospital is based on the subjective, professional assessment of a physician. Each of the records collected from the non-hospitalized individuals was labeled as healthy. Records from the health individuals were collected by conventional techniques not by use of Microsoft Bands. The records generated by the Microsoft Bands from the hospitalized patients were not labeled, but rather were used to test the predictive accuracy of this technique.

The data collected by using the Microsoft Bands was smoothed using a median filter to reduce the effects of outlying data points. For each day (24 hour period of time) the median value out of all of the physiological data values collected that day was used as the single value for that physiological data for that day. For example, if on a given day a Microsoft Band read a patient's resting heart rate three separate times as 180 bpm, 95 bpm, and 90 bpm the median filter would convert those readings into a single reading of 95 bpm for that day. The reading of 180 bpm is likely an artifact introduced by errors from the optical heart rate sensor and this smoothing technique removes the effects of the artifact.

The records for the healthy individuals were used to create a “healthy” dataset referred to herein as H dataset. The records generated by the Microsoft Bands for the hospitalized patients that were labeled as sick by the physician were used as a “sick” dataset referred to herein as S dataset. The records generated by the Microsoft Bands for the hospitalized patients that were labeled as healthy by the physician healthy due to the patients recovering were used as a “recovered” dataset referred to herein as R dataset. The H dataset and the S dataset were both split into two balanced subsets for performing two cross-validations thereby creating H1, H2, S1, and S2 datasets.

The H1 and S1 datasets were combined to create a first training set and the H2 and S2 datasets were combined to create a second training set. The first training set and the second training set were both used as training sets for each of a Gaussian mixture model (GMM), linear discriminant analysis (LDA), and a supported vector machine (SVM). Once trained, the three different machine learning techniques were tested against data that was not included in a given training set. When the first training set (H1+S1) was used H2 and S2 were tested to see how well the machine learning techniques correctly identified the physiological data as corresponding to healthy or sick individuals. Similarly, when the second training set (H2+S2) was used to create models by machine learning, H1 and S1 were used to test the models. Additionally, both models trained by the first training set and by the second training set were tested against records in the R dataset. Results of the tests are provided in Table 1 below.

An additional dataset was collected from 200 healthy individuals in Nebraska referred to herein as N dataset. Physiological data, specifically heart rate and respiration rate, were collecting from these individuals using Microsoft Bands. Records associated with a core temperature greater than 99.5° F. (37.5° C.) were excluded. The core temperature was measured by the user with a conventional thermometer (e.g., under the tongue, in-ear, forehead, etc.) and then manually input to the Microsoft Band. A median filter was applied to this data to exclude outlying data points. The N dataset was used to test the models created by the first (H1+S1) and second (H2+S2) training sets. Thus, the N dataset is an additional set of “healthy” data that was used to validate models trained with entirely separate datasets.

TABLE 1

| | | Healthy (%) | | | Ambiguous (%) | | | Sick (%) | | |
|---|---|---|---|---|---|---|---|---|---|---|
| Training | Testing | GMM | LDA | SVM | GMM | LDA | SVM | GMM | LDA | SVM |
| H1 + S1 | H2 | 41.7 | 41.7 | 25.0 | 58.3 | 58.3 | 75.0 | 0.0 | 0.0 | 0.0 |
| H1 + S1 | S2 | 1.8 | 5.5 | 5.5 | 36.3 | 27.3 | 36.4 | 61.8 | 67.3 | 58.1 |
| H2 + S2 | H1 | 53.8 | 46.2 | 7.7 | 46.2 | 53.8 | 92.3 | 0.0 | 0.0 | 0.0 |
| H2 + S2 | S1 | 0.0 | 4.1 | 4.1 | 62.5 | 45.8 | 45.8 | 37.5 | 50.0 | 50.0 |
| H1 + S1 | R | 11.5 | 42.3 | 23.1 | 69.2 | 50.0 | 65.4 | 19.2 | 7.7 | 11.5 |
| H2 + S2 | R | 11.5 | 30.8 | 23.1 | 76.9 | 57.7 | 65.4 | 11.5 | 11.5 | 11.5 |
| H1 + S1 | N | 54.5 | 96.5 | 94.0 | 45.0 | 0.0 | 0.0 | 0.5 | 3.5 | 6.0 |
| H2 + S2 | N | 61.5 | 92.0 | 86.0 | 38.0 | 0.0 | 43.5 | 0.5 | 8.0 | 10.5 |

Cells containing the most accurate results for a given test are emphasized with bold text. Test for which the tested records are known to be from healthy individuals, specifically datasets H1, H2, R, and N, should return 100% of the individual records as being healthy and 0% as being ambiguous and sick. The better a model is the closer it will come to this result. Both GMM and LDA classified 41.7% of the records from dataset H2 as being healthy. GMM classified 53.8% of the records from dataset H1 as being healthy. LDA trained with the first training set classified 96.5% of the records from N dataset as healthy. When trained with the second training set LDA classified 92% of the records from the N dataset as healthy. All of the models, GMM, LDA, and SVM, correctly identified that none of the records from datasets H1 and H2 represented sick individuals resulting in a sick percentage as 0.0%. The GMM model had the fewest incorrect identifications of records from the N dataset as sick by identifying only 0.5% as being sick when trained with either the first or second training set. However, the GMM model had the greatest difficult classifying records from the N dataset resulting in ambiguous classifications for 45% of the records when trained with the first training set and 38% of the records when trained with the second training set. Thus, the results show that models generated from the first training set and a second training set were able to identify a portion of the records from healthy individuals with a sufficient probability to characterize those records as healthy while the models did not calculate sufficient probability to characterize the remainder of the records as healthy. Thus, these remain records were characterized as ambiguous.

The dataset R which represents records from individuals that have recently recovered from illness was more difficult for the models to classify that the records generated from known healthy individuals in datasets H1 and H2. When testing dataset R, the LDA model created with first training set correctly identified 42.3% of the records as representing healthy individuals and when created with the second training set correctly identified 30.8% of the records as representing healthy individuals. The LDA model created with the first training set had the lowest percentage of mischaracterization of records from the dataset R as sick: 7.7%. When the second training set was used each of the models, GMM, LDA, and SVM, incorrectly characterized 11.5% of the records from dataset R as sick.

When testing a dataset containing records of sick individuals higher percentage of records characterized as sick indicates more accurate performance of a model. For dataset S1, both LDA and SVM models identified 50% of the records as representing sick individuals. However, the GMM model was most accurate with respect to the lowest number of incorrect healthy classifications by returning a result of 0.0% of the records being for healthy individuals. For the tests of dataset S2, the GMM model was also the most accurate with regards to having the minimal number of incorrect healthy classifications; it characterized 1.8% of the records as representing healthy individuals. For dataset S2, the LDA model correctly identified 67.3% of the records as representing sick individuals.

Thus the testing shows that the various models which we trained by machine learning techniques each have different strengths and weaknesses. The testing also shows that the accuracy of a given model varies depending on the records contained in a training set. It is well known to those having skill in the art that a larger set of training data will improve the ability of a trained model to discriminatory between different classes. Thus, a training set containing several thousand or tens of thousands of records—in comparison to the training set containing hundreds of records used in this disclosure—will create models that provide even more accurate classification of physiological records as representing healthy or sick individuals.

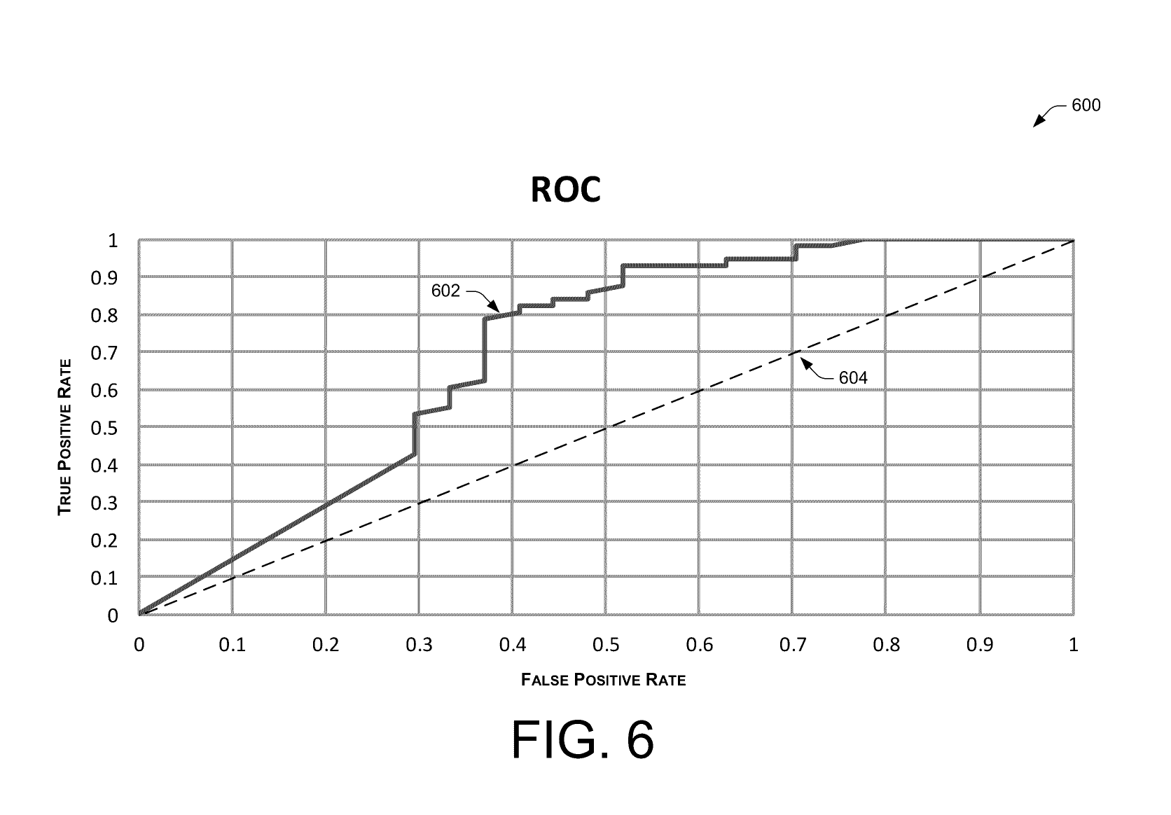

FIG. 6 shows a chart 600 of a receiver operating characteristics (ROC) curve 602 comparing a True Positive Rate for correctly characterizing records from a sick individual as representing sickness with a False Positive rate showing the probability of incorrectly characterizing records from a healthy individual as representing sickness. Presented differently, Prediction Rate=number of records from sick people that the model characterizes as sick/the total number of records from known sick people. And False Alarm=number of records from known healthy individuals that the model predicts to be sick/the total number of records from known healthy individuals. The ROC curve 602 was generated from a LDA model trained with the second training set (H2+S2) used to analyze a set of 68 records including records from 12 healthy individuals (the H2 data) and records from 56 sick individuals (the S2 data).

Line 604 represents the shape of a ROC curve generated by random chance. Thus, the extent that ROC curve 602 is shifted above into the left of line 604 indicates the improved sensitivity (reduced false negatives) and specificity (reduced false positives) of the tested model versus random classification.

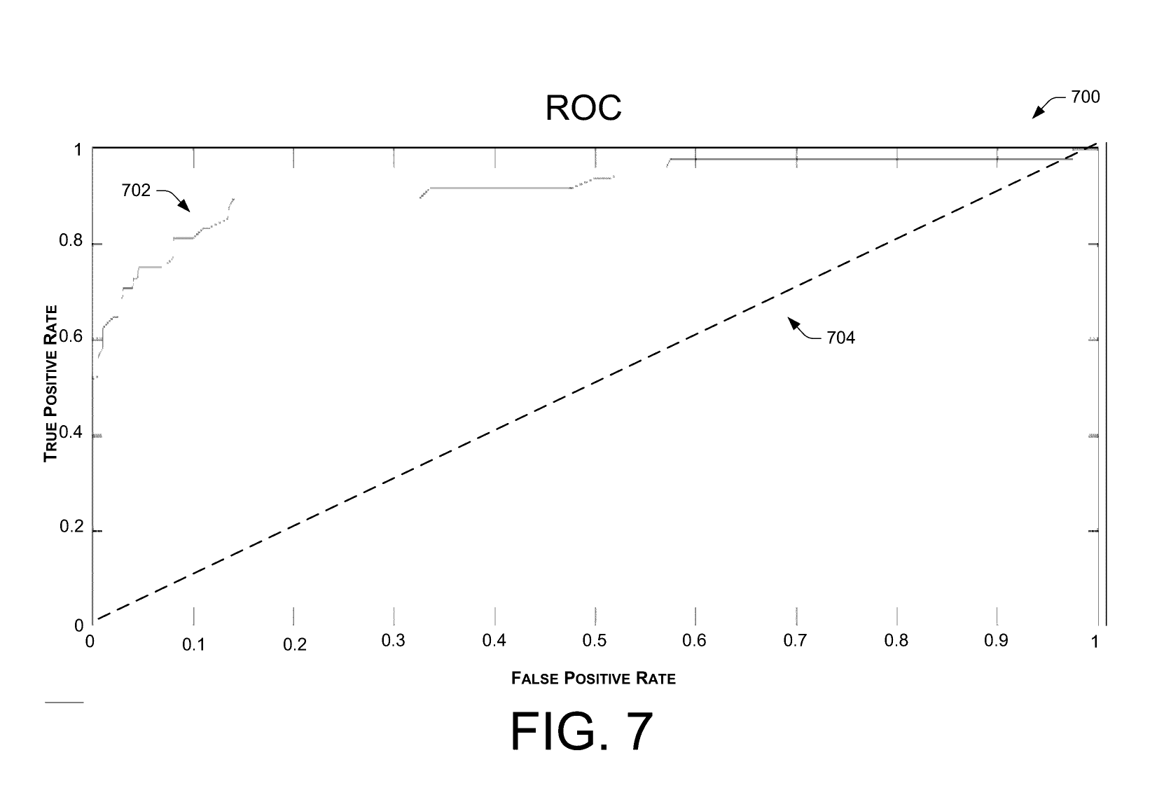

FIG. 7 shows a chart 700 of the ROC curve 702 comparing a True Positive Rate correctly characterizing healthy individuals as healthy with a False Positive Rate showing the probability of incorrectly characterizing records from healthy individuals as representing sickness. The ROC curve 702 was generated from a LDA model trained with the first training set (H1+S1) and used to analyze the 200 records of the N dataset. Line 704 represents the shape of a ROC curve generated by random chance. Thus, the extent that ROC curve 702 is shifted above into the left of line 604 indicates the improved sensitivity (reduced false negatives) and specificity (reduced false positives) of the tested model versus random classification.

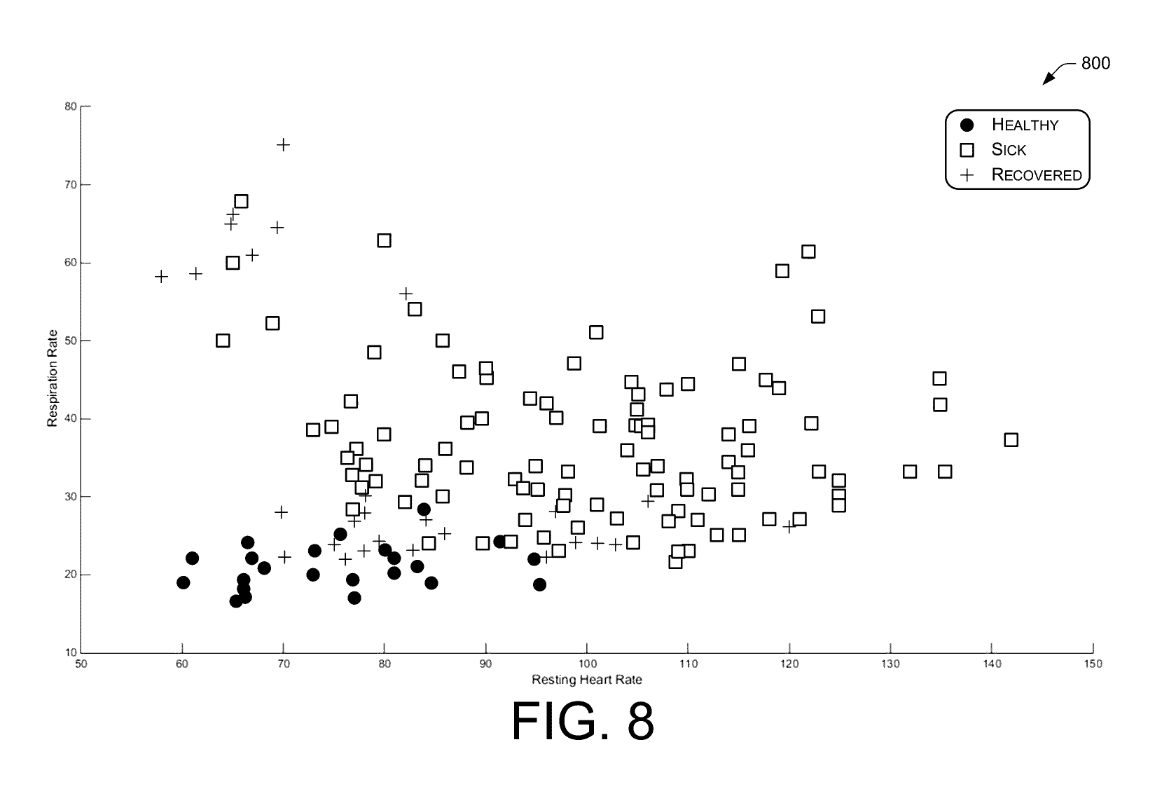

FIG. 8 shows a scatter plot 800 of 151 records from patients where each record includes a respiration rate (vertical axis) and a resting heart rate (horizontal axis). The 151 records includes 23 records from healthy individuals (circles), 101 records from sick individuals (squares), and 27 records from recently recovered individuals (plus signs). The records from the healthy individuals came from the known healthy individuals described above. The records from the sick and recently recovered individuals came from the hospitalized patients and, as described above, a physician manually labeled each record as either indicating a sick patient or a patient who had recovered from illness.

The visualization of the recorded respiration rates and resting heart rates in scatter plot 800 shows the data from the healthy individuals clustered in the lower left which corresponds to relatively lower respiration rates and resting heart rates. Data from the sick individuals spans a range of resting heart rate but generally is associated with respiration rates higher than those of the healthy individuals and tends towards resting heart rates which are also higher than those of the healthy individuals. The data from the recently recovered individuals generally has respiration rates and resting heart rates between that of the healthy and sick individuals. The greater overlap between the values of the records from the recovered individuals and the sick individuals is consistent with the results in Table 1 above which indicate that each of the three models had greater difficulty characterizing records from the dataset R (of recovered individuals) than the test data from either a dataset of healthy individuals or a dataset of sick individuals.

This scatter plot 800 also shows that healthy individuals generally have lower respiration rates and lower resting heart rates while sick individuals usually have elevated respiration rates and elevated resting heart rates. When manually labeling records as corresponding to sick individuals or recently recovered individuals, the physician had access to a more than just heart rate and respiration rate records. Thus, it is notable that respiration rate and resting heart rate alone, even when omitting blood pressure, temperature, and SpO₂, provides sufficient information to characterize a person as being either sick or healthy.

### Illustrative Embodiments

The following clauses described multiple possible embodiments for implementing the features described in this disclosure. The various embodiments described herein are not limiting nor is every feature from any given embodiment required to be present in another embodiment. Any two or more of the embodiments may be combined together unless context clearly indicates otherwise. As used herein in this document. “or” means and/or. For example, “A or B” means A without B, B without A, or A and B. As used herein, “comprising” means including all listed features and potentially including addition of other features that are not listed. “Consisting essentially of” means including the listed features and those additional features that do not materially affect the basic and novel characteristics of the listed features. “Consisting of” means only the listed features to the exclusion of any feature not listed.

Clause 1. A computing system comprising;

one or more processing units;

a memory coupled to the one or more processing units;

one or more network interfaces, configured to be in communication with a wearable electronic device containing one or more physiological sensors;

a physiological data intake module, implemented as instructions executable by the one or more processing units, configured to receive physiological data from the one or more physiological sensors via the one or more network connections and store the physiological data in the memory in association with one or more physiological data descriptors;

a variance detection module, implemented as instructions executable by the one or more processing units the one or more processing units, configured to compare physiological data stored in the memory with one or more baseline values for individual ones of the physiological data descriptors;

a classification module, implemented as instructions executable by the one or more processing units the one or more processing units, which in response to an indication from the variance detection module that the physiological data varies from one or more of the baseline values by more than a threshold amount is configured to input the physiological data into a probabilistic classification model that returns probabilities that the physiological data belongs to one or more classes representing health states; and

a notification module, implemented as instructions executable by the one or more processing units the one or more processing units, configured to send to the wearable electronic device via the one or more network connections a notification based on the health state having a highest probability as determined by the probabilistic classification model.

Clause 2. The system of clause 1, wherein the one or more physiological sensors comprise an optical sensor, the physiological data comprises heart rate data and respiratory rate data, and the physiological data descriptors comprise heart rate and respiratory rate.

Clause 3. The system of clause 1 or 2, wherein the physiological data comprises resting heart rate and respiration rate.

Clause 4. The system of clause 1, 2, or 3, wherein the baseline values for individual ones of the physiological data descriptors are derived from a representative value of the physiological data over a previous period of time.

Clause 5. The system of clause 1, 2, 3, or 4 wherein the probabilistic classification model is one of a mixture model, a discriminant analysis model, or a discriminative model.

Clause 6. The system of clause 1, 2, 3, 4, or 5, wherein the one or more classes representing health states comprise healthy, ambiguous, and sick.

Clause 7. The system of clause 1, 2, 3, 4, 5, or 6, wherein the one or more network connections are in communication with the wearable electronic device via a mobile phone.

Clause 8. A computer-implemented method for detecting a change in respiration rate and heart rate that is indicative of an increased likelihood of developing a fever, the method comprising:

receiving physiological data from a patient, the physiological data comprising respiration rate data, heart rate data, or both;

storing a plurality of time points of the physiological data;

determining that the plurality of time points of the physiological data spans at least a first threshold length of time;

providing the plurality of time points of the physiological data to a probabilistic classification model that returns probabilities that the patient belongs to each of at least two groups, (i) a group that will develop a fever within a second threshold length of time and (ii) a group what will not develop a fever within the second threshold length of time; and

assigning the patient to one of the at least two groups based on the probabilities.

Clause 9. The method of clause 8, wherein physiological data is generated by a wearable electronic device on the patient.

Clause 10. The method of clause 8 or 9, wherein the first threshold length of time is longer than the second threshold length of time.

Clause 11. The method of clause 8, 9, or 10, wherein the first threshold length of time includes at least one period of time during which the patient is resting or asleep.

Clause 12. The method of clause 8, 9, 10, or 11, wherein the probabilistic classification model is created by supervised machine learning from a set of training data including physiological data from a plurality of individuals classified as healthy and a plurality of individuals classified as sick.

Clause 13. The method of clause 12, wherein the set of training data comprises data collected from a plurality of people other than the patient or data previously collected from the patient.

Clause 14. The method of clause 8, 9, 10, 11, or 12, wherein the probabilistic classification model that returns probabilities that the patient belongs to each of at least three groups including, (i) a group that will develop a fever within a second threshold length of time, (ii) a group what will not develop a fever within the second threshold length of time, or (iii) a group that may or may not develop a fever within the second threshold length of time.

Clause 15. A computer-implemented method comprising:

receiving physiological data from a patient;

providing the physiological data to a probabilistic classification model created by machine learning trained with training data including physiological data from a plurality of individuals classified as healthy and a plurality of individuals classified as sick; and

receiving, from the probabilistic classification model, a classification of the health state of the patient as healthy, ambiguous, or sick.

Clause 16. The method of clause 15, wherein the physiological data comprises heart rate data and respiration rate data.

Clause 17. The method of clause 15 or 16, wherein the physiological data is received from an optical sensor included in a device worn by the patient.

Clause 18. The method of clause 15, 16, or 17, wherein the probabilistic classification model is a mixture model, a discriminant analysis model, or a discriminative model.

Clause 19. The method of clause 15, 16, 17, or 18, wherein the receiving the classification comprises receiving a first probability that the patient's health state is correctly classified as healthy and a second probability that the patient's health state is correctly classified as sick and the classification of the patient's health state as healthy, ambiguous, or sick is based on the first probability and the second probability.

Clause 20. The method of clause 15, 16, 17, 18, or 19, further comprising causing a device worn by the patient to display the classification of the patient as healthy, ambiguous, or sick.

Clause 21. A wearable band comprising:

one or more processing units;

a power source coupled to the one or more processing units;

a display coupled to the one or more processing units;

a wireless network connection coupled to the one or more processing units;

an optical sensor coupled to the one or more processing units;

a physiological data sensing module, in communication with the one or more processing units, configured to detect a heart rate and a respiration rate of a wearer of the wearable band by using the optical sensor;

a variance detection module, in communication with the one or more processing units, configured to compare the heart rate and respiration rate with one or more baseline values;

a communication module, in communication with the one or more processing units, that in response to the variance detection module detecting the heart rate or respiration rate exceeding the one or more baseline values is configured to send heart rate data and respiration rate data via the wireless network connection to a remote computing device;

a notification module, in communication with the one or more processing units, configured to receive a probability of the wearer developing a fever from the remote computing device and to cause the display to present a representation of the probability.

Clause 22. A computing system comprising:

means for processing;

mean for storing information;

means for communication via a network;

means for detecting a physiological data;

means for receiving physiological data from the means for detecting physiological data via the means for communicating via the network;

means for storing the physiological data in the means for storing information in association with one or more physiological data descriptors;

means for comparing the physiological data stored in the means for storing information with one or more baseline values for individual ones of the physiological data descriptors;

responsive to the means for comparing determining that the physiological data varies from one or more of the baseline values by more than a threshold, means for inputting the physiological data into a probabilistic classification model that returns probabilities that the physiological data belongs to one or more classes representing health states; and

means for sending to the wearable electronic device a notification based on the health state having a highest probability as determined by the probabilistic classification model via the means for communication via a network.

### CONCLUSION

Although the subject matter has been described in language specific to structural features and/or methodological acts, it is to be understood that the subject matter defined in the appended claims is not necessarily limited to the specific features or acts described above. Rather, the specific features and acts are disclosed as example forms of implementing the claims.

1. A computing system comprising;
one or more processing units;
a memory coupled to the one or more processing units;
one or more network interfaces, configured to be in communication with a wearable electronic device containing one or more physiological sensors;
a physiological data intake module, implemented as instructions executable by the one or more processing units, configured to receive physiological data from the one or more physiological sensors via the one or more network connections and store the physiological data in the memory in association with one or more physiological data descriptors;
a variance detection module, implemented as instructions executable by the one or more processing units the one or more processing units, configured to compare physiological data stored in the memory with one or more baseline values for individual ones of the physiological data descriptors;
a classification module, implemented as instructions executable by the one or more processing units the one or more processing units, which in response to an indication from the variance detection module that the physiological data varies from one or more of the baseline values by more than a threshold amount is configured to input the physiological data into a probabilistic classification model that returns probabilities that the physiological data belongs to one or more classes representing health states; and
a notification module, implemented as instructions executable by the one or more processing units the one or more processing units, configured to send to the wearable electronic device via the one or more network connections a notification based on the health state having a highest probability as determined by the probabilistic classification model.

2. The system of claim 1, wherein the one or more physiological sensors comprise an optical sensor, the physiological data comprises heart rate data and respiratory rate data, and the physiological data descriptors comprise heart rate and respiratory rate.

3. The system of claim 1, wherein the physiological data comprises resting heart rate and respiration rate.

4. The system of claim 1, wherein the baseline values for individual ones of the physiological data descriptors are derived from a representative value of the physiological data over a previous period of time.

5. The system of claim 1, wherein the probabilistic classification model is one of a mixture model, a discriminant analysis model, or a discriminative model.

6. The system of claim 1, wherein the one or more classes representing health states comprise healthy, ambiguous, and sick.

7. The system of claim 1, wherein the one or more network connections are in communication with the wearable electronic device via a mobile phone.

8. A computer-implemented method for detecting a change in respiration rate and heart rate that is indicative of an increased likelihood of developing a fever, the method comprising:
receiving physiological data from a patient, the physiological data comprising respiration rate data, heart rate data, or both;
storing a plurality of time points of the physiological data;
determining that the plurality of time points of the physiological data spans at least a first threshold length of time;
providing the plurality of time points of the physiological data to a probabilistic classification model that returns probabilities that the patient belongs to each of at least two groups, (i) a group that will develop a fever within a second threshold length of time and (ii) a group what will not develop a fever within the second threshold length of time; and
assigning the patient to one of the at least two groups based on the probabilities.

9. The method of claim 8, wherein physiological data is generated by a wearable electronic device on the patient.

10. The method of claim 8, wherein the first threshold length of time is longer than the second threshold length of time.

11. The method of claim 8, wherein the first threshold length of time includes at least one period of time during which the patient is resting or asleep.

12. The method of claim 8, wherein the probabilistic classification model is created by supervised machine learning from a set of training data including physiological data from a plurality of individuals classified as healthy and a plurality of individuals classified as sick.

13. The method of claim 12, wherein the set of training data comprises data collected from a plurality of people other than the patient or data previously collected from the patient.

14. The method of claim 8, wherein the probabilistic classification model that returns probabilities that the patient belongs to each of at least three groups including, (i) a group that will develop a fever within a second threshold length of time, (ii) a group what will not develop a fever within the second threshold length of time, or (iii) a group that may or may not develop a fever within the second threshold length of time.

15. A computer-implemented method comprising:
receiving physiological data from a patient;
providing the physiological data to a probabilistic classification model created by machine learning trained with training data including physiological data from a plurality of individuals classified as healthy and a plurality of individuals classified as sick; and
receiving, from the probabilistic classification model, a classification of the health state of the patient as healthy, ambiguous, or sick.

16. The method of claim 15, wherein the physiological data comprises heart rate data and respiration rate data.

17. The method of claim 15, wherein the physiological data is received from an optical sensor included in a device worn by the patient.

18. The method of claim 15, wherein the probabilistic classification model is a mixture model, a discriminant analysis model, or a discriminative model.

19. The method of claim 15, wherein the receiving the classification comprises receiving a first probability that the patient's health state is correctly classified as healthy and a second probability that the patient's health state is correctly classified as sick and the classification of the patient's health state as healthy, ambiguous, or sick is based on the first probability and the second probability.

20. The method of claim 15, further comprising causing a device worn by the patient to display the classification of the patient as healthy, ambiguous, or sick.
